(19) United States
(12) Patent Application Publication
(10) Pub. No.: US 2009/0287239 A1
(43) Pub. Date: Nov. 19, 2009

(54) Tissue Bulking Device and Method

(75) Inventor: Shawn M. Wignall, Woodbury, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/467,900

(22) Filed: May 18, 2009

Related U.S. Application Data
(60) Provisional application No. 61/054,028, filed on May 16, 2008.

(51) Int. Cl. A61M 29/00; A61M 5/178

(52) U.S. Cl. 606/191; 604/160

(57) ABSTRACT

Tissue bulking devices and methods of using such devices for restricting a flow of fluid through a patient's body lumen, such as a urethral lumen or rectal lumen as a way alleviating incontinence. The bulking device has a first collapsed state for deliver and a second expanded state to displace a portion of the lumen thereby reducing its inner diameter and restricting fluid flow. The bulking device generally has a plurality of openings providing access to an interior thereof for promoting tissue ingrowth, thereby further securing the device in its location and aiding in tissue bulking. Support members attached to the bulking device further aid in tissue ingrowth and bulking.

### PRIORITY

The present application claims the benefit of U.S. provisional application No. 61/054,028 filed on May 16, 2008.

### BACKGROUND OF THE INVENTION

### Field of the Invention

The present invention relates to treating incontinence and particularly to a method of and apparatus for delivering a tissue bulking device into a target tissue structure generally proximate to an outer surface of a lumen wall.

As set forth in commonly assigned U.S. Pat. No. 6,964,699, urinary incontinence is a significant clinical problem and a major source of disability and dependency. The most frequently occurring types of urinary incontinence are stress incontinence, urge incontinence, overflow incontinence, and mixed incontinence.

Stress incontinence is a common form of incontinence in men and women. When intra-abdominal pressure exceeds urethral pressure upon coughing, sneezing, laughing, lifting, or like activity, it can cause leakage of urine. Known causes of stress incontinence include physical changes associated with pregnancy, childbirth, and menopause.

Urge incontinence, another form of urinary incontinence, occurs when a patient loses urine while suddenly feeling the urge to urinate. The patient is unable to inhibit the flow of urine long enough to reach the toilet. Inappropriate bladder contractions are the most common cause of urge incontinence, and may occur in connection with central nervous system lesions, urinary infection, or bladder tumors, to name several examples.

Another form of urinary incontinence is overflow incontinence, which occurs when the bladder is unable to empty normally. Weak bladder muscles, which can be caused by nerve damage from diabetes, or a blocked urethra, caused by tumors or urinary stones, are among the more common causes of overflow incontinence.

Many options are available to treat incontinence in its various forms, including Kegel exercises, electrical stimulation, biofeedback, timed voiding or bladder training, medications, pessaries, implantation of urethral slings to support the urethra, invasive or minimally invasive surgery, catheterization, and other methods and devices.

One additional option for treating urinary incontinence involves periurethral or transurethral injection of a bolus of biocompatible bulk-enhancing or bulking agent into a tissue structure around the urethra including the urethral wall, the bladder neck, bladder suspension ligaments, the urethral sphincter, pelvic ligaments, pelvic floor muscles, fascia, and the like. The injected bulking material adds bulk to the tissue structure to restore and maintains continence, as described, for example, in U.S. Pat. No. 7,014,607 and in U.S. Patent Application Publication No. 2005/0288639. For example, the bulking agent is injected in or adjacent the urethral sphincter muscle in the area of the urethro-vesical junction, where the urethra extends from the bladder, to increase pressure on and reduce the size of the urethral lumen, thereby providing resistance to the flow of urine. A transurethral injection is made by entering the urethra and penetrating through the urethral wall, whereas a periurethral injection is made through an external skin and passing through the tissues surrounding the urethra to dispose the injecting instrument tip in tissue structure in the area of the urethro-vesical junction.

Typical bulking agents include inert Teflon® plastic particles, autologous fat, collagen, and polyhydroxyalkanoate materials. In addition, U.S. Pat. No. 6,702,731 discloses biocompatible, tissue-reactive polymers that bond with tissue to form a bulk polymer in-situ that is biocompatible, elastomeric and non-biodegradable. The bonded polymer remains in place and does not substantially change volume over time.

The periurethral and transurethral injections of such bulking masses are typically done employing a cystoscope to provide direct visualization of the urethra and can be completed on an outpatient basis. However, the physician must be trained in the use of the cystoscope, and its use entails employing relatively expensive cystoscopic equipment in an operating or procedure room and use of anesthesia. The physician must undergo extensive training on how to precisely inject bulking mass with cystoscopic visualization.

A transurethral bulking mass injection procedure employing a specialized instrument adapted to be inserted through the urethra to function as an injection guide and needle stop is disclosed in U.S. Patent Application Publication No. 2005/0288639 and asserted to obviate the need for the cystoscope and specialized surgical training.

In another option, a detachable balloon can be placed in tissue in the area of the urethro-vesical junction immediately adjacent to the urethra and inflated to function as a bulking mass. The inflated balloon provides pressure external to the urethra, which causes partial closure of the urethra and reduces unwanted fluid leakage from the bladder through the urethra. Depending upon the target site location and/or physician preference, the balloon delivery system can be used in parallel with a viewing instrument or can be passed through a lumen of a viewing instrument.

For example, in the periurethral delivery, the deflated balloon and balloon delivery instrument are advanced through the patient's tissue parallel to the urethra to the target site for delivery and inflation of the balloon. The desired target site and correct positioning of the assembly are observed using a viewing instrument passed through, for example, the urethra. In the transurethral delivery, the deflated balloon and balloon delivery instrument are inserted through the lumen of a viewing instrument such as a cystoscope, and the assembly is advanced through the urethral wall to the target site.

In still another treatment option disclosed in U.S. Pat. No. 6,976,492 and in U.S. Patent Application Publication No. 2005/0288544, thermal heating or cooling energy is applied to induce controlled shrinkage or contraction of a support tissue structure, typically being a collagenated tissue such as fascia, ligament, or the like. For treatment of urinary incontinence, the tissue structure is responsible in some manner for control of urination or for supporting such a tissue, e.g. the urethral sphincter.

Disclosed systems include a urethral guide having a distal balloon adapted to be inserted in the urethra to dispose the balloon in the bladder for inflation therein and a vaginal guide adapted to be inserted into the vagina. The urethral and vaginal guides include treatment delivery surfaces, e.g., RF electrodes or other heating or cooling surfaces that face one another so that heating energy or cooling is concentrated in the tissue structure disposed therebetween.

A urethral measurement assembly facilitates registration of a treatment delivery surface with a fractional location along the urethral axis, such as the mid-point of the urethra. Ideally, the physician will have some freedom to move a treatment probe manually as desired to achieve the best thermal contact, electrical contact, ergonomic fit to the patient, or the like, while maintaining registration with sufficient tolerances or within an acceptable registration region. The registration region may be established so as to avoid inadvertent damage to nerves or other tissues, which may result from treatments outside, and particularly beyond the desired axial range of, the registration region.

The above mentioned devices and methods have achieved some success in alleviating some forms of incontinence. The devices and methods, unfortunately, have several shortcomings, including shrinkage and migration within the body in the case of bulking agents and deflation in the case of balloon tissue bulking devices.

### BRIEF SUMMARY OF THE INVENTION

The present invention is generally directed to tissue bulking devices and methods for the treatment of incontinence that resolves some of the aforementioned shortcomings of the prior art. In one embodiment at least one bulking device is introduced transvaginal or transperineal (for males) and positioned adjacent to or alongside an outer surface of a lumen such as a patient's urethra to effect at least partial constriction of the lumen. A similar approach is also available for treatment of anal or fecal incontinence by introducing the bulking device near or under the rectum to approximate the ano-rectal angle.

In at least one aspect of the invention the bulking member has a first collapsed configuration or state for delivery adjacent to the lumen. The bulking member also has a second expanded deployed configuration or state. In the second expanded configuration the bulking member displaces at least a portion of the lumen from its normal anatomical position thereby reducing an inner diameter of the lumen and thus restricting a flow of fluid.

In another aspect of the invention the bulking member may include one or more openings or apertures permitting access to an interior of the bulking member. The openings permit tissue ingrowth into the member once implanted. The tissue ingrowth aids in tissue bulking and securing the bulking member adjacent to the outer surface of the lumen.

One or more tissue support members can be attached to the bulking member to aid in tissue ingrowth. The support members can comprise fibers, mesh, sutures and the like. The tissue support members can be attached to the outside, inside or both of the bulking member.

The above summary of the invention is not intended to describe each illustrated embodiment or every implementation of the invention. The figures in the detailed description that follow more particularly exemplify these embodiments.

### BRIEF DESCRIPTION OF THE DRAWINGS

The invention will be better understood when consideration is given to the following detailed description thereof. Such description makes reference to the annexed drawings wherein:

FIG. 1A is a perspective view of a tissue bulking member in an expanded configuration having two closed opposed ends.

FIG. 1B is a perspective view of another tissue bulking member in an expanded configuration having one open and one closed opposed ends.

FIG. 1C is a perspective view of another tissue bulking member in an expanded configuration having two open opposed ends.

FIG. 1D is a perspective view of another tissue bulking member in a generally smaller configuration.

FIG. 1E is a perspective view of another tissue bulking member in an expanded configuration.

FIG. 1F is a cross section along line 1F-1F of FIG. 1E.

FIG. 2A is an illustration of a pelvis having a bladder, urethra, vagina, uterus and rectum.

FIG. 2B is an illustration of a pelvis having a bladder, urethra, and sphincter muscles.

FIG. 3 is a perspective view of a tissue bulking member in a collapsed state.

FIG. 4A is a partial cross section view of a tissue bulking member disposed in a channel of a delivery tool.

FIG. 4B is a partial cross section view of a tissue bulking member disposed in a channel of a delivery tool having an example deployment mechanism.

FIG. 4C is a partial cross section view of a tissue bulking member disposed in a channel of a delivery tool that is used in conjunction with a cystoscope.

FIG. 5 is a perspective view of a delivery tool delivering tissue bulking member to a therapeutic location.

FIG. 6 is a perspective view of multiple tissue bulking members positioned in therapeutic locations. a piston attached to a bracket of the door.

The preceding description of the drawings is provided for example purposes only and should not be considered limiting. The following detailed description is provided for more detailed examples of the invention. Other embodiments not disclosed or directly discussed are also considered to be within the scope and spirit of the invention. It is not the intention of the inventor to limit the scope of the invention by describing one or more example embodiments.

### DESCRIPTION OF EXAMPLE EMBODIMENTS

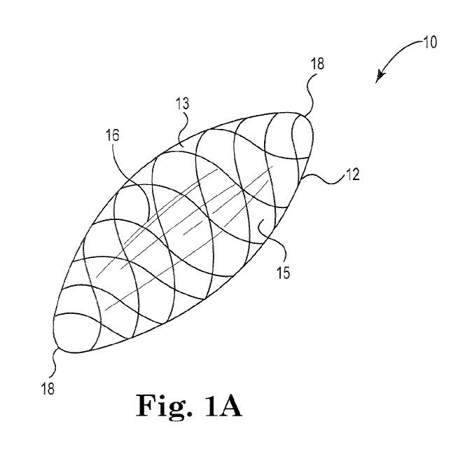

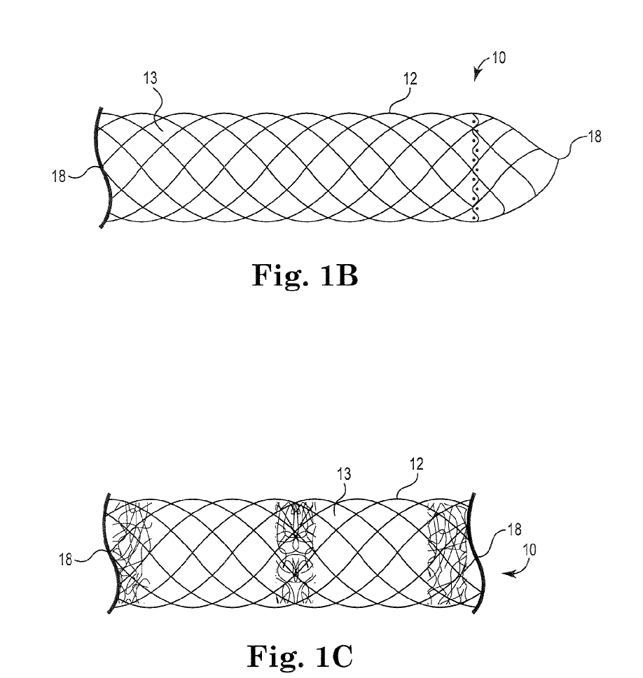

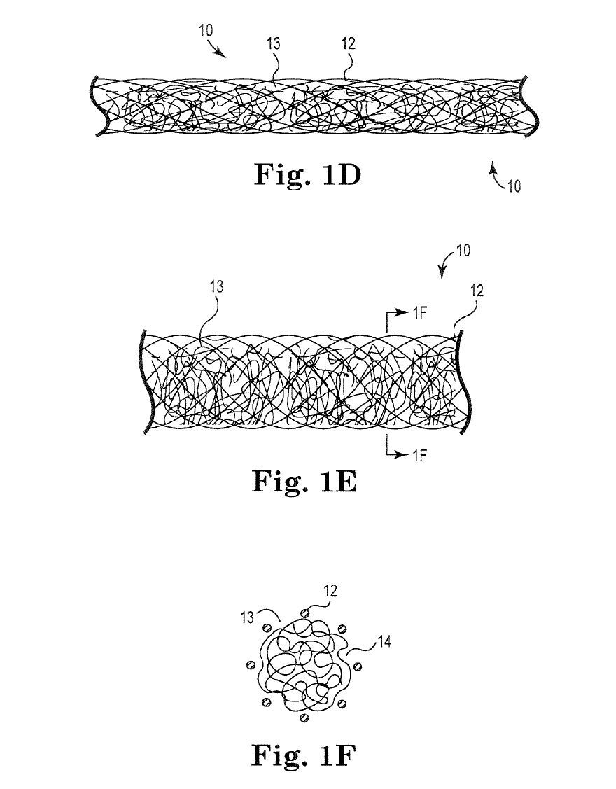

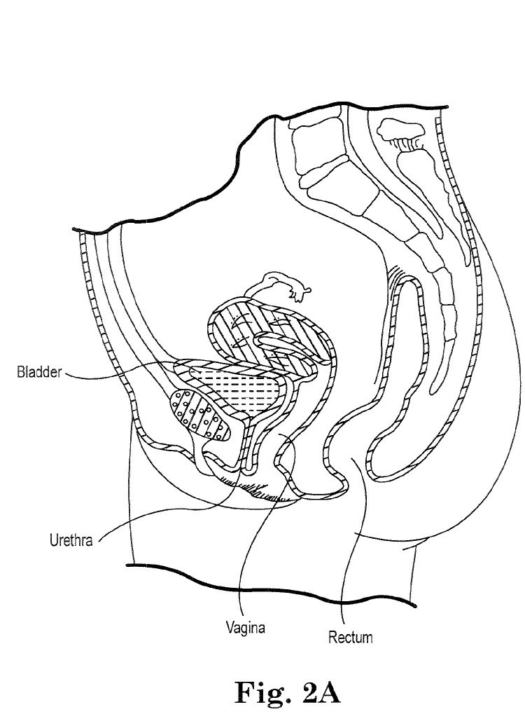

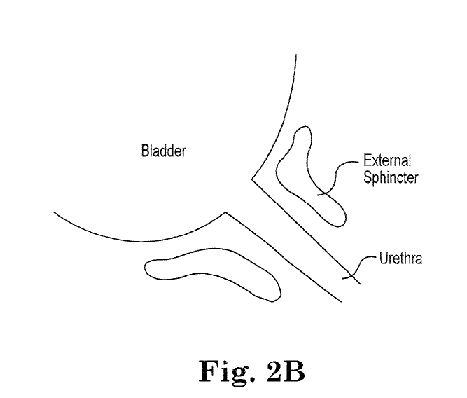

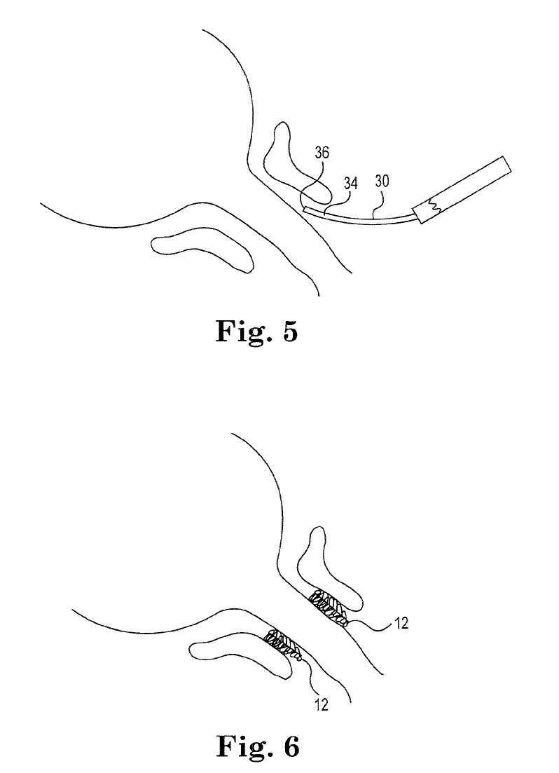

The invention, indicated by the numeral 10 in example FIGS. 1-6, is directed to a device to restrict a flow of a fluid through a lumen in order to treat various types of incontinence. In some example embodiments, the device 10 comprises at least one tissue bulking member 12 that is capable of being inserted or implanted into a therapeutic location adjacent to an outer surface of a lumen to be constricted.

In one example embodiment of the invention, as illustrated in FIG. 1A, the tissue bulking member 12 comprises a wall, frame or lattice-work having a plurality of openings 13 that provide access to an interior 15 of the tissue bulking member 12. The openings 13 of the tissue bulking member 12 can be formed from a frame made from a single sheet of material or threads or wires of material woven together. The tissue bulking member 12 can be manufactured from a polymer, metal or any other type of biologically compatible material.

Although the structure of the tissue bulking member 12, unlike typical bulking agents, reduces its ability to move or migrate once implanted, the openings 13 make the tissue bulking member 12 permeable to tissue ingrowth into the interior 15 of the tissue bulking member 12 to further ensure that migration does not occur. Additionally, as tissue ingrowth occurs it may be possible for the newly formed tissue to assert forces upon the outer surface of the adjacent lumen and assist in controlling fluid flowing through it and thus incontinence.

In some embodiments, as illustrated in FIGS. 1A-1C-1F, one or more tissue support members 16 may be positioned in the interior 15 of the tissue bulking member 12 to further support tissue ingrowth. The tissue support members 16 may be positioned in a particular location, as illustrated in the example embodiment of FIG. 1C, or it can be positioned along an entire length of the tissue bulking member 12 as illustrated in FIGS. 1A-1E. In example embodiments, the tissue support members 16 comprise individual strands of a biologically compatible material such a polypropylene. In other example embodiments, the strands can be woven together and possibly attached to the tissue bulking member 12. In yet other example embodiments, the strands of the tissue support members 16 can be attached to an outer surface of the tissue bulking member 12 or interweave between the interior 15 and exterior of the tissue bulking member 12 via the openings 13. Numerous configurations for positioning the tissue support members 16 in relation to the tissue bulking member 12 are possible and should be considered to be within the spirit and scope of the present invention.

In one example embodiment, as illustrated in FIG. 1A, the tissue bulking member 12 may have a generally tubular shape or configuration with closed opposed ends 18 that may assist in its delivery to a therapeutic location. As illustrated in FIG. 1B the opposed ends 18 of the tissue bulking member 12 can be generally open thereby providing additional opportunity for tissue ingrowth into the interior 15. As illustrated in FIG. 1B, the opposed ends 18 may have one opened and one closed. The generally closed end may assist the tissue bulking member 12 during delivery to a therapeutic location while the open end provides additional opportunity for tissue ingrowth once implanted.

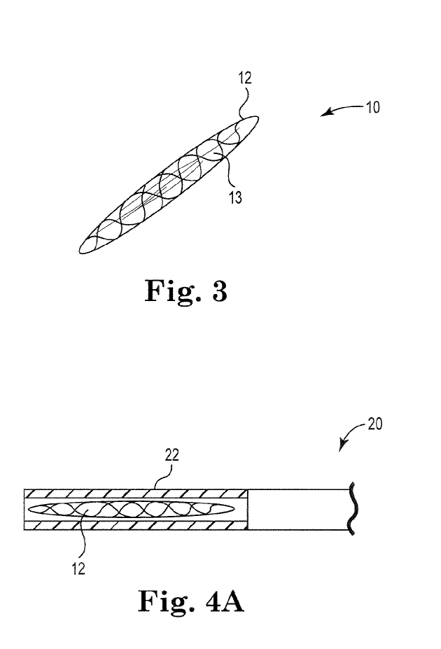

As illustrated in FIGS. 1D and 3 the tissue bulking member 12 has a first collapsed configuration for delivery adjacent to an outer surface of a lumen. In its first collapsed configuration the interior 15 has a generally reduced diameter, which aids in its placement adjacent the lumen. In embodiments having tissue support members 16, it is preferable that the tissue support members 16 be generally pliable or compressible to permit the tissue bulking member 12 to collapse.

As illustrated in FIGS. 1A-1C, and 1E, tissue bulking member 12 has a second expanded configuration for exerting a force upon an outer surface of an adjacent or proximate lumen. As particularly illustrated in FIG. 1A, the amount of outwardly radiating expansion can vary along a length of the tissue bulking member 12. If the opposed ends 18 are closed an inner diameter of the tissue bulking member 12 may generally taper toward the opposed ends 18. If the opposed ends 18 are open the inner diameter of the tissue bulking member 12 may be generally uniform along its length. It is also possible to have a generally spherical tissue bulking member 12.

The tissue bulking member 12 may be self-expanding once it is implanted. In some embodiments of the invention the woven strands of the tissue bulking member 12 permit the tissue bulking member 12 to be collapsible but self-expanding. In some embodiments of the invention, the tissue bulking member 12 may be manufactured from a material that has memory properties and transitions between two states or configurations upon the application of heat, light, magnetism and the like. Such materials include, but are not limited to, nitinol and the like.

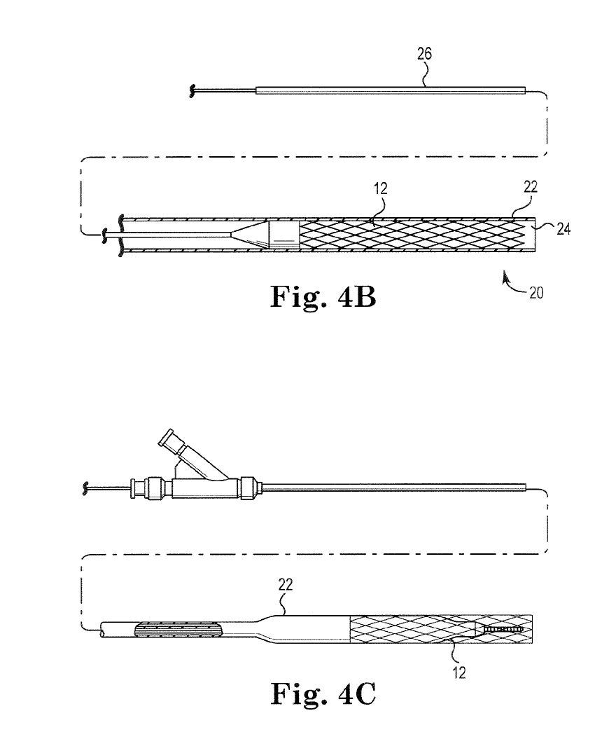

In order to deliver the tissue bulking member 12 to a therapeutic location a delivery device or tool 20, as illustrated in FIGS. 4A-4C, may be provided separately or as a kit with one or more tissue bulking members 12. As particularly illustrated in FIGS. 4B and 4C, a delivery tool 20 may comprise a catheter 22 having a lumen 24 capable of housing the bulking member or members 12. The tissue bulking member 12 can be generally deformed into a smaller configuration within the lumen 24 of the catheter 22. A deployment member 26 can axially transverse the lumen 24 of the catheter 22 and push or deploy the tissue bulking member 12 out an end of the lumen 24 that is either already opened or can be opened. When the tissue bulking member 12 is deployed it may automatically expand from the smaller configuration to a larger configuration. As it transitions from the smaller configuration toward the larger configuration it exerts a force against the lumen displacing a portion of the lumen and restricting the flow of fluid though it.

In another embodiment, the delivery tool 20 may comprise a needle 30 mounted on a handle 32. The needle 30 may have an interior 34 adjacent a distal end 36 opposite the handle 32 to house the tissue bulking member 12 and tissue support member(s) 16. When the tissue bulking member 12 is in the interior 34 it may be in a smaller configuration. As with the catheter 22 described above, the needle 30 may include a deployment member (not shown) that can be used to deploy the tissue bulking member 12 from the interior 34 to a therapeutic location adjacent to an outer surface of a lumen to restricted. In either embodiment the deployment member 22 may comprise a rod that can transverse the interior 34 and engage the tissue bulking member 12. The deployment member 22 may be attached to an actuator such as a slideable or depressible button on the handle. Any deployment member or mechanism may be used to move the tissue bulking member 12 to the therapeutic location from the delivery tool 20.

In yet another example embodiment of the invention, a system or kit is provided to restrict a flow of a fluid through a lumen such as a urethra or a rectum. The system may comprise a delivery tool 20 having a proximal end and a distal end. The distal end may have an axially extending channel or lumen 34 extending at least partially from the distal end toward the proximal end. One or more bulking members 12 may be disposed in the channel or lumen 34 during the manufacturing process or they may be manually loaded at the time of the procedure. One or more tissue support members 16 can be attached to the bulking member 12 as described above to aid in tissue ingrowth into the tissue bulking member 12. As described above, the delivery tool 20 may comprise a catheter 22 or a needle 30. Both types of delivery tools 20 may include a deployment mechanism such as a push rod and the like to deploy the tissue bulking device 12 to a therapeutic location

In use, a physician locates a therapeutic location, which may include a location between an outer surface of a urethra and the sphincter muscle surrounding the urethra. Once the therapeutic location has been identified the physician may make a stab incision either in the vagina for a female or proximate the perineal for a male. The physician may dissect to the therapeutic location to create a pathway for the delivery tool 20.

Once the therapeutic location has been located and a pathway established the physician may insert the distal end of the catheter 22 or needle 30 through the stab incision and follow the pathway to the therapeutic location. Once in the therapeutic location the physician may activate a deployment mechanism to deliver the tissue bulking member 12, which is contained within the catheter 22 or needle 30 in a first collapsed state or smaller configuration, to the therapeutic location.

Once the tissue bulking member 12 is deployed it may automatically expand to a second expanded state or larger configuration. As it expands it exerts a force against an outer surface of lumen and the sphincter muscle. The force causes at least partial displacement of the lumen causing a restricted flow of fluid through it. If the lumen needs to be restricted further the above process may be repeated placing additional tissue bulking members 12 circumferentially around the outer surface of the lumen.

Once the tissue bulking member 12 is deployed tissue cells migrate through the apertures 13 causing tissue ingrowth. The migrating cells may be supported by the tissue support members 16 attached to the tissue bulking member 12. The new ingrowth may further secure the tissue bulking member 12 in place and may provide additional force upon the outer surface of the lumen being restricted.

The invention may be embodied in these and other specific forms without departing from the spirit or attributes thereof, and it is therefore desired that the embodiments be considered in all respects as illustrative and not restrictive, reference being made to the appended claims rather than to the foregoing description to indicate the scope of the invention.

What is claimed is:

1. A device to restrict a flow of a fluid through a lumen, the device comprising:
at least one bulking member having a plurality of openings permitting tissue ingrowth into an interior of the bulking member when positioned outside and adjacent to a lumen to be restricted; and
a tissue support member disposed in the interior of the bulking member to support tissue ingrowth, wherein tissue ingrowth into the bulking member permanently restricts the flow of fluid through the lumen.

2. A device according to claim 1, in which the bulking member has a generally tubular shape with opened opposed ends.

3. A device according to claim 1, in which the bulking member has a generally tubular shape with closed opposed ends.

4. A device according to claim 1, in which the bulking member has a generally tubular shape with one open end and one closed end.

5. A device according to claim 1, in which the bulking member is has a first collapsed state for delivery adjacent to an outer surface of the lumen to be restricted and a second expanded state for restricting the lumen and permitting tissue ingrowth.

6. A device according to claim 1, in which the support member comprises a plurality of fibers capable of supporting tissue ingrowth.

7. A device according to claim 1, further comprising a delivery tool capable of carrying the bulking member and support member adjacent to the lumen.

8. A device according to claim 7, in which the delivery tool comprises a catheter having a lumen capable of housing the bulking member in a first collapsed state adjacent to the lumen, wherein upon deployment from the catheter the bulking member moves from the first collapsed state to the second expanded state thereby displacing a portion of the lumen and restricting the flow of fluid therethrough.

9. A device according to claim 7, in which the delivery tool comprises a needle mounted on a handle, the needle having an interior adjacent a distal end opposite the handle to house the bulking member and support member in a first collapsed state, wherein upon deployment from the needle the bulking member moves from the first collapsed state to the second expanded state, thereby displacing a portion of the lumen and to restricting the flow of fluid therethrough.

10. A device according to claim 1, in which a plurality of bulking members are disposed about an outer surface of a lumen to displace at least a portion of the lumen and restrict a flow of fluid therethrough.

11. A system to restrict a flow of a fluid through a lumen, the system comprising:
a delivery tool having a proximal end and a distal end, the distal end having an axially extending channel extending at least partially therein, the distal end being positionable adjacent an outer surface of a lumen;
a bulking member disposed in the channel of the delivery tool in a first collapsed state and upon deployment from the delivery tool adjacent the outer surface of the lumen expands to a second collapsed state; and
a tissue support member attached to the bulking member to aid in tissue ingrowth into the bulking member.

12. A system according to claim 11, in which the delivery tool comprises a surgical needle having a deployment mechanism to deploy the bulking member.

13. A system according to claim 11, in which the delivery tool comprises a catheter having a deployment mechanism to deploy the bulking member.

14. A system according to claim 11, in which the bulking member has a generally tubular shape having a plurality of openings providing access to an interior thereof for tissue cell ingrowth.

15. A system according to claim 11, in which the tissue support member comprises a plurality of fibers disposed in an interior of the bulking member to aid in tissue ingrowth.

16. A method of restricting a flow of fluid through a lumen, the method comprising the steps of:
inserting adjacent to an outer surface of a lumen at least one bulking member having a plurality of openings providing access to an interior thereof; and
expanding the bulking member from a first collapsed state to a second expanded state adjacent the outer surface of the lumen such that the bulking member displaces at least a portion of the lumen to restrict fluid flowing therethrough;

17. A method according to the claim of 16, further comprising the step of tissue ingrowth into the interior of the bulking thereby securing the bulking adjacent to the outer surface of the lumen.

18. A method according to the claim of 17 further includes attaching tissue support members to the bulking member to support tissue ingrowth into the interior of the bulking.

19. A method according to the claim of 16, in which the step of inserting the bulking member comprises using a delivery tool to insert the bulking member adjacent to an outer surface of a lumen.

20. A method according to the claim of 16 further comprising the step of inserting additional bulking members adjacent to an outer surface of a lumen to restrict a flow of fluid therethrough.
